(19) United States
(12) Patent Application Publication
(10) Pub. No.: US 2006/0084170 A1
(43) Pub. Date: Apr. 20, 2006

(54) Cytokine-free growth and maintenance of progenitor cells

(76) Inventors: Mark J Pykett, Boxford, MA (US); Michael Rosenzweig, Boston, MA (US); Todd M Upton, Chestnut Hill, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/515,075

(22) PCT Filed: May 23, 2003
(86) PCT No.: PCT/US03/16419

§ 371 Date: Dec. 5, 2005

Related U.S. Application Data
(60) Provisional application No. 60/383,239, filed on May 24, 2002.

(51) Int. Cl. C12N 5/08

(52) U.S. Cl. 435/372

(57) ABSTRACT

The invention pertains to methods and devices for the in vitro culture of hematopoietic progenitor cells in the absence of exogenously added hematopoietic growth factors. The hematopoietic progenitor cells are cultured in the absence of exogenously added hematopoietic growth factors without loss in cell progenitor cell numbers and/or functionality, while maintaining progenitor cell pluripotency.

### FIELD OF THE INVENTION

This invention relates generally to hematopoietic cells, and more specifically to methods for in vitro culturing of hematopoietic progenitor cells.

### BACKGROUND OF THE INVENTION

The circulating blood cells, such as erythrocytes, leukocytes, platelets and lymphocytes, are the products of the terminal differentiation of recognizable precursors. In fetal life, hematopoiesis occurs throughout the reticular endothelial system. In the normal adult, terminal differentiation of the recognizable precursors occurs exclusively in the marrow cavities of the axial skeleton, with some extension into the proximal femora and humeri and vertebrae. These precursor cells, in turn, derive from very immature cells, called progenitors, which are assayed by their development into contiguous colonies of mature blood cells in 1-3 week cultures in semi-solid media, such as methylcellulose.

Human bone marrow cell cultures initially were found to have a limited hematopoietic potential, producing decreasing numbers of hematopoietic progenitor and mature blood cells, with cell production ceasing by six to eight weeks. Subsequent modifications of the original system resulted only in minor improvements. This has been largely attributed to the dependence of the hematopoietic progenitor cells upon environmental influences such essential growth factors (hematopoietic growth factors and cytokines) found in vivo (see, e.g., U.S. Pat. Nos. 5,599,703, 5,728,851, and 6,372,210).

Previous efforts to advance in vitro proliferation and differentiation of hematopoietic progenitor cells, examined the effects of cytokines in various substrates, including pre-seeded stroma and fibronectin. The addition of exogenous growth factors to the culture environment, particularly IL-3 (Interleukin-3) and GM-CSF (Granulocyte Macrophage-Colony Stimulating Factor), may lead to selective expansion of only specific lineages. These findings suggest that addition of exogenous growth factors into hematopoietic progenitor cell cultures may adversely affect the multipotency of primitive hematopoietic progenitor cells by causing them to differentiate and thus depleting the immature hematopoietic progenitor population.

Alternative approaches have used irradiated bone marrow stroma to culture and support hematopoietic progenitor cells and have been shown to maintain these progenitor cells as long-term culture initiating cells (LTCICs) (which are immature cells) and to increase transduction of hematopoietic progenitor cells and LTCICs by retroviral vectors. However, questions have been raised about the risks of infection and immune reaction to transplantation of non-autologous bone marrow. Fibronectin, a cellular stromal component, reduces this risk of infection and immune mediated response while enhancing retroviral transduction. However, fibronectin alone may not be sufficient to maintain primitive hematopoietic progenitor cells in vitro.

Hematopoietic progenitor cell expansion for bone marrow transplantation is a potential application of human long-term bone marrow cultures. Human autologous and allogeneic bone marrow transplantation are currently used as therapies for diseases such as leukemia, lymphoma, and other life-threatening diseases. For these procedures, however, a large amount of donor bone marrow must be removed in an attempt to obtain enough cells for engraftment, and even such efforts often do not yield adequate cell numbers.

An approach providing hematopoietic progenitor cell expansion would reduce the need for large bone marrow donation and would make possible obtaining a small marrow donation and then expanding the number of progenitor cells in vitro before infusion into the recipient. Also, it is known that a small number of hematopoietic progenitor cells circulate in the blood stream. If these cells could be selected and expanded, then it would be possible to obtain the required number of hematopoietic progenitor cells for transplantation from peripheral blood and eliminate the need for bone marrow donation.

Hematopoietic progenitor cell expansion would also be useful to aid recovery from chemotherapy and radiation treatment and is another application for human long-term bone marrow cultures. Most chemotherapy agents and radiation act by killing cells going through cell division. Bone marrow is one of the most prolific tissues in the body and is therefore often the organ that is initially damaged by chemotherapy drugs and radiation. The result is that blood cell production is rapidly destroyed during such treatment, which often must be terminated to allow the hematopoietic system to replenish the blood cell supplies before a patient is re-treated with chemotherapy.

A successful approach providing hematopoietic progenitor cell expansion would greatly facilitate the production of a large number of non-differentiated precursor cells and further differentiated precursor cells of a specific lineage, and in turn provide a larger number of differentiated hematopoietic cells with a wide variety of applications, including blood transfusions.

There exists a need to influence favorably hematopoietic progenitor cell viability and pluripotency under culture in vitro.

There exists a need to provide large numbers of differentiated hematopoietic cells.

An object of the invention is to provide methods for the expansion and proliferation of hematopoietic stem cells while maintaining the hematopoietic progenitor cell properties of self-renewal and pluripotency.

Another object of the invention is to provide methods for the controlled production in large numbers of specific lineages of progenitor cells and their more differentiated hematopoietic cells. These and other objects of the invention will be described in greater detail below.

### SUMMARY OF THE INVENTION

The invention, in one important part, involves improved methods for culturing hematopoietic progenitor cells, which methods can, for example, maintain the pluripotency and self-renewal capabilities of hematopoietic progenitor cells. Thus, one aspect of the invention is improved preservation of a culture of hematopoietic progenitor cells. Another aspect is an improvement in the number of progeny that can be obtained from a sample of hematopoietic progenitor cells. Still another aspect of the invention is an improvement in the number of differentiated progeny blood cells that can be obtained from a sample of hematopoietic progenitor cells.

Surprisingly, according to the invention, it has been discovered that hematopoietic progenitor cells can be cultured without the addition of exogenous growth factors, which prevents the induction of differentiation and/or the loss of progenitor cells during culture. Thus, the present invention permits the culture of hematopoietic progenitor cells in vitro without adding hematopoietic growth factors, inoculated stromal cells or stromal cell conditioned medium. This is achieved, simply, by culturing the hematopoietic progenitor cells in a medium containing only serum. Such results were never before realized using known art methodologies (e.g., as in U.S. Pat. No. 5,677,139 by Johnson et al., which describes the in vitro differentiation of CD3⁺ cells on primate thymic stroma monolayers, or as in U.S. Pat. No. 5,541,107 by Naughton et al., which describes a three-dimensional bone marrow cell and tissue culture system, or as in U.S. Pat. No. 6,372,210 by Brown which describes a serum-free medium that supports the proliferation and differentiation of CD34+ cells but which requires the addition of exogenous factors to maintain the immature phenotype of the cells).

According to one aspect of the invention, a method for in vitro culture of hematopoietic progenitor cells is provided. An amount of hematopoietic progenitor cells is introduced to a culture chamber. The cells are cultured in an environment that is free of inoculated stromal cells, stromal cell conditioned medium, and exogenously added hematopoietic growth factors that promote differentiation, other than serum.

The hematopoietic progenitor cells may be derived from a tissue such as bone marrow (including unfractionated bone marrow), peripheral blood (including mobilized peripheral blood), umbilical cord blood, placental blood, fetal liver, embryonic cells (including embryonic stem cells), aortal-gonadal-mesonephros derived cells, and lymphoid soft tissue. Lymphoid soft tissue includes the thymus, spleen, liver, lymph node, skin, tonsil and Peyer's patches.

In other embodiments, the method further includes the step of harvesting hematopoietic cells. There may be a first harvesting after a first culturing period. There may be at least one additional harvesting after at least one additional culturing period. The harvested cells may then be cultured in at least one of an exogenously added agent selected from the group consisting of a hematopoietic growth factor that promotes hematopoietic cell maintenance, expansion and/or differentiation and influences cell localization, inoculated stromal cells, and stromal cell conditioned medium. In certain embodiments, the hematopoietic growth factor that promotes hematopoietic cell maintenance, expansion and/or differentiation, and influences cell localization, may be an agent that includes interleukin 3, interleukin 6, interleukin 7, interleukin 11, interleukin 12, stem cell factor, FLK-2 ligand/FLT-3 ligand, Epo, Tpo, GMCSF, GCSF, Oncostatin M, and/or MCSF.

According to any of the foregoing embodiments, the method of the invention can include, in said first culturing step, culturing the cells in an environment that is free of hematopoietic progenitor cell survival and proliferation factors such as interleukin 3, interleukin 6, interleukin 7, interleukin 11, interleukin 12, stem cell factor, FLK-2 ligand/FLT-3 ligand, Epo, Tpo, GMCSF, GCSF, Oncostatin M, and MCSF. As mentioned above, the inventors have discovered, surprisingly, that hematopoietic progenitor cells can be grown without the addition of any of these agents which typically are added in the prior art in order to prevent the hematopoietic progenitor cells from dying and/or differentiating during culture and which were thought to be required to cause cell proliferation so as to increase the number of stem cells. Still another embodiment of the invention is performing the first culturing step in an environment that is free altogether of any exogenously added hematopoietic progenitor cell growth factors (including cytokines), other than serum.

As will be understood, according to the invention, it is possible now to culture hematopoietic progenitor cells for 7, 8, 9, 10 days, or up to and including 14 days without the addition of exogenous growth factors.

According to the invention, it is also possible now to culture hematopoietic progenitor cells without the induction of differentiation and/or the loss of progenitor cells during culture, and to harvest the cells during this time interval for subsequent exposure to culture conditions containing hematopoietic growth factors that promote hematopoietic cell maintenance, expansion and/or differentiation, and/or introducing them into a subject. Culturing and harvesting over this time period is an independent aspect of the invention.

According to another aspect of the invention, a method is provided for in vitro culture of hematopoietic progenitor cells to produce differentiated cells of hematopoietic origin. In a first culturing step, a first amount of hematopoietic progenitor cells is cultured in an environment that is free of inoculated stromal cells, stromal cell condition medium and exogenously added hematopoietic growth factors that promote hematopoietic cell maintenance, expansion and/or differentiation, other than serum, under conditions and for a period of time to increase the number of cultured hematopoietic progenitor cells relative to said first amount or to increase the functionality of the hematopoietic progenitor cells, thereby producing a second amount of hematopoietic progenitor cells. Then, in a second culturing step, at least a portion of the second amount of cultured hematopoietic progenitor cells is cultured in an environment that includes at least one of an agent selected from the group consisting of a hematopoietic growth factor that promotes hematopoietic cell maintenance, expansion and/or differentiation, inoculated stromal cells and stromal cell conditioned medium, to produce differentiated cells of hematopoietic origin. In one embodiment, the environment is free of hematopoietic growth factors that promote survival and proliferation of hematopoietic progenitor cells such as interleukins 3, 6 and 11, Tpo, stem cell factor and FLK-2 ligand/FLT-3 ligand. In another embodiment, the environment of the first culturing step is free of any hematopoietic growth factors other than those present as a result of the addition of serum to the nutritive medium. In this aspect of the invention, the method further can comprise a second culturing step which is a plurality of second culturing steps, each comprising culturing only a portion of the second amount of hematopoietic progenitor cells. The method also can involve a harvesting step between the first and second culturing steps, wherein the harvesting step comprises harvesting the at least a portion of the second amount prior to culturing the at least a portion of the second amount in the second culturing step. The harvesting step also can be a plurality of harvesting steps spaced apart in time and, in this instance, the second culturing step can be a plurality of second culturing steps, one for each of the harvesting steps. The preferred source of the hematopoietic progenitor cells and the culture conditions are as described above.

In any of the foregoing embodiments involving hematopoietic cell maintenance, expansion and/or differentiation using a hematopoietic growth factor, the hematopoietic growth factor used is selected from the group consisting of interleukin 3, interleukin 6, interleukin 7, interleukin 11, interleukin 12 stem cell factor, FLK-2 ligand/FLT-3 ligand, Epo, Tpo, GMCSF, GCSF, Oncostatin M, and MCSF.

These and other aspects of the invention are described in greater detail below.

### BRIEF DESCRIPTION OF THE FIGURES

FIG. 1 is a histogram of the average total cell number in a seven day cytokine-free stem cell expansion culture.

FIG. 2 is a histogram of average cell viability in a seven day cytokine-free stem cell expansion culture.

FIG. 3 is a histogram of the average number of CD34+ cells in a seven day cytokine-free stem cell expansion culture.

FIG. 4 is a histogram of the average percent of CD34+ cells in a seven day cytokine-free stem cell expansion culture.

It is to be understood that the figures are not required for enablement of the invention.

### DETAILED DESCRIPTION OF THE INVENTION

The invention in one aspect involves culturing hematopoietic progenitor cells in an environment that is free altogether of inoculated stromal cells, stromal cell conditioned medium, and exogenously added hematopoietic growth factors that promote hematopoietic cell maintenance, expansion and/or differentiation, other than serum.

The cells cultured according to the methods of the invention are hematopoietic progenitor cells. “Hematopoietic progenitor cells” as used herein refers to immature blood cells having the capacity to self-renew and to differentiate into the more mature blood cells (also described herein as “progeny”) comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), lymphocytes (e.g. B cells, T cells, NK cells), antigen presenting cells (e.g. dendritic cells, Kupfer cells, Langerhans cells) and monocytes (e.g., circulating monocytes, tissue macrophages, microglia). It is known in the art that such cells may or may not include CD34⁺ cells. CD34⁺ cells are immature cells present in the “blood products” described below, express the CD34 cell surface marker, and are believed to include a subpopulation of cells with the “progenitor cell” properties defined above. Preferred cells according to the invention include AC133 antigen-expressing cells (see, e.g., U.S. Pat. No. 5,843,633), and/or CD34⁺ cells. Hematopoietic progenitor cells may also include cell types not traditionally thought to possess hematopoietic potential that have recently been shown to be able to form blood cells. Such cells have recently been isolated from brain, liver, muscle, and other tissue sources. (Bjornson C R, et al., Science, 1999, 283(5401):534-7; Gritti A, et al., J Physiol, 2002, 96(1-2):81-90. Review; Muench M O, et al., J Immunol, 2001, 167(9):4902-9; Weissman I L, Science, 2000, 287(5457):1442-6. Review).

The hematopoietic progenitor cells can be obtained from blood products. A “blood product” as used in the present invention defines a product obtained from the body or an organ of the body containing cells of hematopoietic origin. Such sources include unfractionated bone marrow, umbilical cord, peripheral blood, liver, thymus, lymph and spleen (all of which can be mobilized). It will be apparent to those of ordinary skill in the art that all of the aforementioned crude or unfractionated blood products can be enriched for cells having “hematopoietic progenitor cell” characteristics in a number of ways. For example, the blood product can be depleted from the more differentiated progeny. The more mature, differentiated cells can be selectively removed, via cell surface molecules they express. Additionally, the blood product can be fractionated selecting for CD34⁺ cells and/or AC133⁺ cells. As mentioned earlier, CD34⁺ cells are thought in the art to include a subpopulation of cells capable of self-renewal and pluripotentiality. Such selection can be accomplished using, for example, commercially available magnetic anti-CD34 beads (Dynal, Lake Success, N.Y.), and/or anti-AC133 beads (Miltenyi Biotec, Auburn, Calif.). Unfractionated blood products can be obtained directly from a donor, retrieved from cryopreservative storage, and/or a commercial supplier (e.g., Poietics, Gaithersburg, Md.).

Employing the culture conditions described in greater detail below, it is possible according to the invention to preserve hematopoietic progenitor cells and to stimulate the expansion of hematopoietic progenitor cell number and/or colony forming unit potential. Once expanded, the cells, for example, can be returned to the body to supplement, replenish, etc. a patient's hematopoietic progenitor cell population. This might be appropriate, for example, after an individual has undergone chemotherapy. There are certain genetic conditions wherein hematopoietic progenitor cell numbers are decreased, and the methods of the invention may be used in these situations as well.

It also is possible to take the increased numbers of hematopoietic progenitor cells produced according to the invention and stimulate them with hematopoietic growth agents that promote hematopoietic cell maintenance, expansion and/or differentiation, to yield the more mature blood cells, in vitro. Such expanded populations of blood cells may be applied in vivo as described above, or may be used experimentally as will be recognized by those of ordinary skill in the art. Such differentiated cells include those described above, as well as T cells, plasma cells, erythrocytes, megakaryocytes, basophils, polymorphonuclear leukocytes, monocytes, macrophages, eosinophils and platelets, and their respective direct precursors.

In certain embodiments of the invention, the hematopoietic progenitor cells are continuously cultured and the cultured cells are harvested. “Harvesting hematopoietic cells” is defined as the dislodging or separation of cells from the culture chamber. This can be accomplished using a number of methods, such as enzymatic, centrifugal, electrical or by size, or the one preferred in the present invention, by incubating the cells with Cell Dissociation Solution (BioWhittaker, Walkersville, Md.). The cells can be further collected and separated. “Harvesting steps spaced apart in time” or “intermittent harvest of cells” is meant to indicate that a portion of the cells are harvested, leaving behind another portion of cells for their continuous culture in the established media, maintaining a continuous source of the original cells and their characteristics. Harvesting “at least a portion of” means harvesting a subpopulation of or the entirety of. Thus, as will be understood by one of ordinary skill in the art, the invention can be used to expand the number of hematopoietic progenitor cells, all the while harvesting portions of those cells being expanded for treatment to develop even larger populations of differentiated cells.

A “culture chamber,” as used herein, refers to plastic dishes, roller bottles, and plastic (e.g., polypropylene) bags, commonly used in the art. In certain embodiments, three-dimensional matrices are specifically excluded from the scope of culture chambers according to the present invention.

In all of the culturing methods according to the invention the media used is that which is conventional for culturing cells, for example RPMI, DMEM, ISCOVES, etc., supplemented with an effective amount of fatty acid, an effective amount of cholesterol, an effective amount of transferrin (or an effective amount of an iron salt), and insulin in an amount of 0.25 to 2.5 U/ml (or an effective amount of insulin like growth factor). Media containing such supplements are commercially available (e.g., from Quality Biological, Inc., Gaithersburg, Md.), and/or are described in U.S. Pat. No. 6,372,210 B2 to Brown). A preferred, supplemented medium according to the present invention is QBSF (Quality Biological, Inc., Gaithersburg, Md.). Importantly, media used according to the invention are supplemented with human or animal serum, preferably human if the hematopoietic progenitor cells are also of human origin. Serum at 2%-5% concentration in the media is preferred, although lesser (e.g., less than 0.05%, less than 0.1%, less than 0.25%, less than 0.5%, less than 0.75%, less than 1.0%, less than 1.5%, and any integer therebetween as if explicitly recited herein) or greater concentrations may be used. When used at these concentrations, serum can contain small amounts of hematopoietic growth factors naturally found in the serum. The serum is preferably autologous but can be pooled. “Autologous”, as used herein, refers to material obtained from the subject from which the hematopoietic progenitor cells (in culture) originated. Serum albumin (human or animal) may also be included in the media. According to the invention, culture medium can be added (supplement), partially replaced (e.g., of equal volume), or left unchanged during the culture of cells of the invention.

The growth agents of particular interest in connection with the present invention are hematopoietic growth factors. By hematopoietic growth factors, it is meant factors that influence the survival, proliferation or differentiation of hematopoietic cells. Growth agents that affect only survival and proliferation, but are not believed to promote differentiation, include the interleukins 3, 6 and 11, stem cell factor, and FLK-2 ligand/FLT-3 ligand. Hematopoietic growth factors that promote differentiation include the colony stimulating factors such as GMCSF, GCSF, MCSF, Tpo, Epo, Oncostatin M, and interleukins other than IL-3, 6 and 11. The foregoing factors are well known to those of ordinary skill in the art. Most are commercially available. They can be obtained by purification, by recombinant methodologies or can be derived or synthesized synthetically.

In one aspect of the invention, the cells according to the invention are cultured in an environment that is free of exogenously added cytokines (“cytokine-free”). “Cytokine” is a generic term for soluble proteins which are released from one cell subpopulation and which act as intercellular mediators, for example, in the generation or regulation of an immune response. See Human Cytokines: Handbook for Basic & Clinical Research (Aggrawal, et al. eds., Blackwell Scientific, Boston, Mass. 1991) (which is hereby incorporated by reference in its entirety for all purposes). Cytokines include, e.g., interleukins IL-1 through IL-15, tumor necrosis factors α & β, interferons α, β, and γ, tumor growth factor beta (TGF-β), colony stimulating factor (CSF) and granulocyte monocyte colony stimulating factor (GM-CSF). The action of each cytokine on its target cell is mediated through binding to a cell surface receptor. Cytokines share many properties of hormones, but are distinct from classical hormones in that in vivo, they generally act locally on neighboring cells within a tissue.

In another aspect of the invention, the cells according to the invention are cultured in an environment that is free of inoculated stromal cells, stromal cell conditioned medium and exogenously added hematopoietic growth factors that promote differentiation of hematopoietic cells, other than serum. By “free of inoculated stromal cells”, it is meant that the cell culture chamber is free of stromal cells which have been independently introduced into the chamber as an inoculum for promoting survival, proliferation or differentiation of the hematopoietic progenitor cells, excluding, however, stromal cells which are contained naturally in the isolate blood product and which may survive and proliferate in culture upon inoculation of the isolate blood product.

“Stromal cells” as used herein comprise fibroblasts and mesenchymal cells, with or without other cells and elements, that can be used to establish conditions that favor the subsequent attachment and growth of hematopoietic progenitor cells. Fibroblasts can be obtained via a biopsy from any tissue or organ, and include fetal fibroblasts. These fibroblasts and mesenchymal cells may be transfected with exogenous DNA that encodes, for example, one of the hematopoietic growth factors described above.

“Stromal cell conditioned medium” refers to medium in which the aforementioned stromal cells have been incubated. The incubation is performed for a period sufficient to allow the stromal cells to secrete factors into the medium. Such “stromal cell conditioned medium” can then be used to supplement the culture of hematopoietic progenitor cells promoting their proliferation and/or differentiation.

Thus, when cells are cultured without any of the foregoing agents, it is meant herein that the cells are cultured without the addition of such agent except as may be present in serum, ordinary nutritive media or within the blood product isolate, unfractionated or fractionated, which contains the hematopoietic progenitor cells.

The culture of the hematopoietic cells preferably occurs under conditions to increase the number of such cells and/or the colony forming potential of such cells. The conditions used refer to a combination of conditions known in the art (e.g., temperature, CO₂ and O₂ content, nutritive media, etc.). The time sufficient to increase the number of cells is a time that can be easily determined by a person skilled in the art, and can vary depending upon the original number of cells seeded. As an example, discoloration of the media can be used as an indicator of confluency. Additionally, and more precisely, different volumes of the blood product can be cultured under identical conditions, and cells can be harvested and counted over regular time intervals, thus generating the “control curves”. These “control curves” can be used to estimate cell numbers in subsequent occasions. According to the present invention, a preferred time for culturing the hematopoietic cells is 7 days. Although this period can be extended by a few days, Applicants discovered that by day 14 both the total number of cells and the number of progenitor cells is reduced when compared to the numbers of cells at 7 days under the specified conditions.

The conditions for determining colony forming potential are similarly determined. Colony forming potential is the ability of a cell to form progeny. Assays for this are well known to those of ordinary skill in the art and include seeding cells into a semi-solid medium, treating them with growth factors and counting the number of colonies.

As used herein, a subject is a human, non-human primate, cow, horse, pig, sheep, goat, dog, cat or rodent. Human hematopoietic progenitor cells and human subjects are particularly important embodiments. According to the invention, an amount of the cells is introduced in vitro into a cell culture chamber, and cultured in an environment that is free of inoculated stromal cells, stromal cell conditioned medium, and exogenously added hematopoietic growth factors that promote hematopoietic cell maintenance, expansion and/or differentiation, other than serum.

The invention will be more fully understood by reference to the following examples. These examples, however, are merely intended to illustrate the embodiments of the invention and are not to be construed to limit the scope of the invention.

### EXAMPLES

Cell Separation and Culture:

CD34⁺ hematopoietic progenitor cells were derived from mononuclear cells isolated from human mobilized peripheral blood (mPB) by Ficoll separation and magnetic anti-human CD34⁺ beads (Miltenyi Biotec, Auburn, Calif.).

CD34+ hematopoietic progenitor cells can also be derived from human bone marrow or umbilical cord blood. These sources are commercially available from Poietics, Gaithersburg, Md. In some instances, the magnetic bead separation step can be followed by separation from the beads using an anti-idiotype antibody (e.g., Detachabead, Dynal).

Five hundred thousand CD34+ cells were seeded into individual wells of a standard 48-well tissue culture plate (Becton Dickinson/Falcon, Bedford, Mass.). Cultures utilized between 0.35-1 ml of QBSF-60 liquid medium (Quality Biological, Gaithersburg, Md.) supplemented with 5% pooled human AB serum (BioWhittaker, Walkersville, Md.). Cultures were incubated in a 37° C., 5% CO₂ incubator for 7-14 days.

After the culture period, all non-adherent cells were harvested from each well, counted and surface antigen stained. Cell numbers and viability were determined by Trypan Blue exclusion and enumeration on a Nuebauer Haemocytometer.

Results:

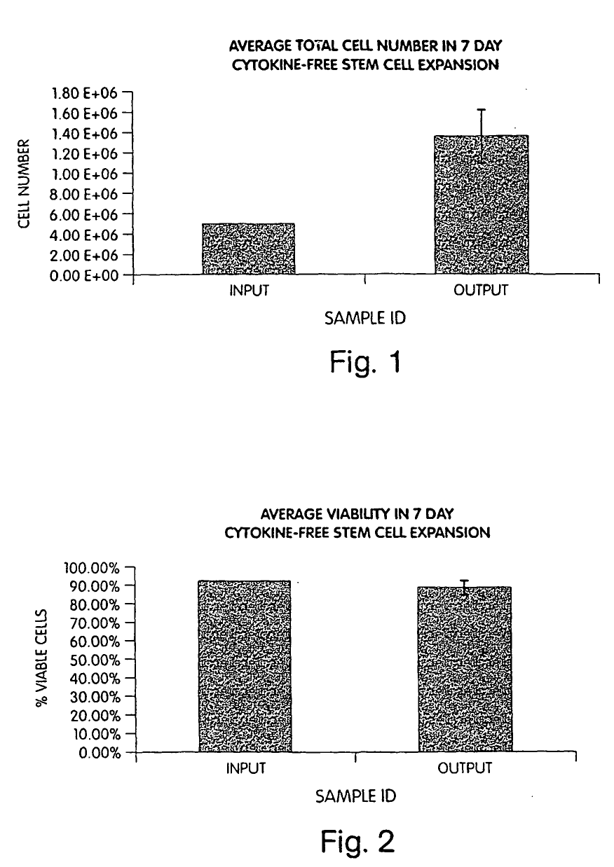

Average cell number at harvest was 1.36 million viable cells. This is a 2.6 fold increase in cell number over input. The viability of cells at harvest (89.1%) was found to be nearly identical to the viability at input (92.3%). These results are shown in FIGS. 1 and 2.

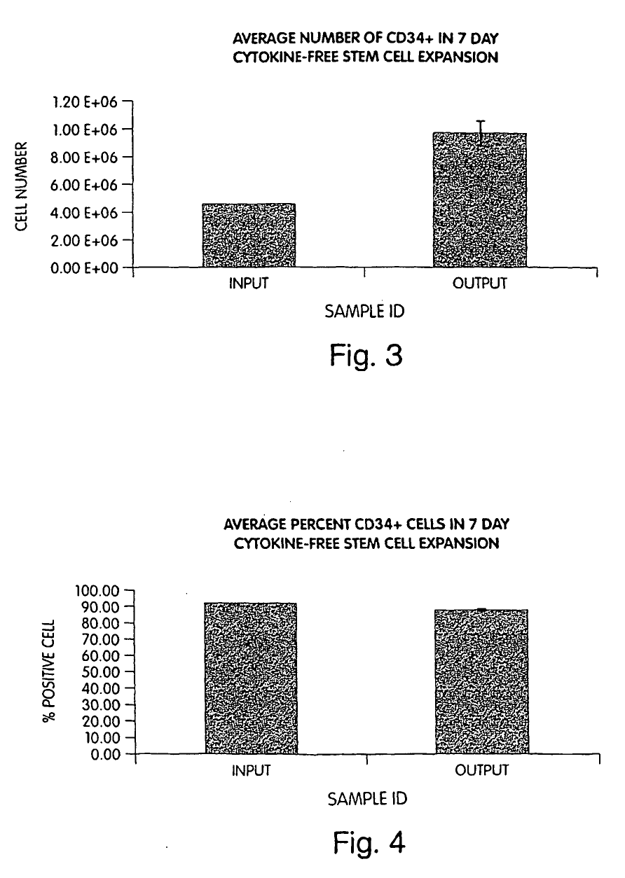

Similar results are seen when the CD34+ population is examined. The average number of CD34+ cells at harvest was 979,000. This represents a 2.1 fold increase over input. The average percent CD34 marker positive cells was similar in both the input and output populations (92.0% v. 87.5%). These results are shown in FIGS. 3 and 4.

Antibodies used for surface phenotype determination will include anti-CD34 (Qbend10, Beckman/Coulter, Brea, Calif.), anti-CD38 and anti-CD45 (both BD Immunocytometry, San Diego, Calif.) antibodies to evaluate progenitor cell distributions. Flow cytometry analysis of the cells was performed using multi-parameter FACScan flow cytometry analysis using a FACSCalibur instrument (Becton Dickinson). Appropriate controls included matched isotype antibodies to establish positive and negative quadrants, as well as appropriate single color stains to establish compensation. For each sample, at least 10,000 list mode events were collected.

In Vitro Assays of Hematopoietic Progenitors Cells Harvested from Cytokine-Free Culture:

The ability of HPCs (cultured as described above for 7-14 days) to produce myeloid and erythroid colonies can be demonstrated using traditional methylcellulose assays. An exemplary methylcellulose assay is described below, however one of ordinary skill in the art will be able to modify the assay as necessary without undue experimentation.

Equal numbers of cells isolated from cultures as described above are added at a density of 1.33×10⁴ cells/ml to 3.5 ml of methylcellulose medium with cytokines (IL-3 20 ng/ml; GM-CSF 30 ng/ml; erythropoietin 3 IU/ml; stem cell factor 50 ng/ml; all Stem Cell Technologies, Vancouver, Calif.), plus 0.5 ml of DMEM (2% FCS, 10 IU/ml penicillin, 10 μg/ml streptomycin, 1 mM L-glutamine). 1.5 ml of this mixture is added to a scored petri dish using a syringe and a blunt needle to avoid bubbles. Duplicate assays are performed for each condition. Two duplicate petri dishes are then placed in an incubator with 5% CO₂ at 37° C. for 10-21 days. After 10-21 days, the number of colonies are determined by manual counting. Positive colonies are scored on the basis of an accumulation of 20 or more cells. Erythroid colonies are scored after 14-21 days on the basis of a gold-brown pigment, demonstrating hemoglobin, whereas myeloid colonies are identified by their predominantly transparent appearance. Counts are performed in duplicate.

In Vivo Assays of Hematopoietic Progenitors Cells Harvested from Cytokine-Free Culture:

The proliferative and differentiative potential of cultured HPCs (cultured as described above) can also be demonstrated in vivo using animal models known in the art. These in vivo assays demonstrate the ability of HPCs to produce multiple types of blood cell progeny (i.e. multipotency), to self-renew, and/or to engraft in a host. One such model system is the sublethally-irradiated, immunodeficient, nonobese diabetic-scid/scid (NOD/SCID) mouse (Conneally E, et al., Proc Natl Acad Sci USA, 1997, 94:9836-41). Briefly, HPCs cultured according to the afore-mentioned methods of the invention are injected intravenously to a sublethally-irradiated, immunodeficient, NOD/SCID mouse, and the bone marrow of such recipients is examined 6 to 8 weeks post-transplant for engraftment (e.g., by using limiting dilution analysis to measure the frequency of cells that produce both CD34CD19+ (B-lymphoid) and CD34+ (myeloid) colony-forming cell progeny).

### Equivalents

Those skilled in the art will recognize, or be able to ascertain using no more than routine experimentation, many equivalents to the specific embodiments of the invention described herein. Such equivalents are intended to be encompassed by the following claims. All references disclosed herein are incorporated by reference in their entirety.

We claim:

1. A method for in vitro culture of hematopoietic progenitor cells comprising:
introducing an amount of hematopoietic progenitor cells into a culture chamber, and
culturing said cells in an environment that is free of inoculated stromal cells, stromal cell conditioned medium, and exogenously added hematopoietic growth factors that promote hematopoietic cell maintenance, expansion and/or differentiation, other than serum.

2. The method of claim 1, wherein the environment is free of interleukins 3, 6 and 11, Tpo, stem cell factor and FLT/FLK ligand growth factors.

3. The method of claim 1, wherein the environment is free of hematopoietic growth factors.

4. The method of claim 1, further comprising:
before said introducing step, obtaining said hematopoietic progenitor cells from a blood product.

5. The method of claim 4, wherein said blood product is mobilized peripheral blood or mobilized umbilical cord blood.

6. The method of claim 1, wherein the hematopoietic progenitor cells are cultured under conditions and for a time sufficient to increase the number of hematopoietic progenitor cells relative to the amount introduced into said culture chamber.

7. The method of claim 1, further comprising:
after said culturing step, harvesting the cells.

8. The method of claim 7, further comprising:
culturing said harvested hematopoietic cells in at least one of an exogenously added agent selected from the group consisting of a hematopoietic growth factor that promotes hematopoietic cell maintenance, expansion and/or differentiation, inoculated stromal cells and stromal cell conditioned medium.

9. The method of claim 8, further comprising:
culturing hematopoietic cells obtained from said first harvesting in the presence of an exogenously added agent, and
culturing hematopoietic cells obtained from said at least one additional harvesting in the presence of an exogenously added agent,
wherein said exogenously added agent is selected from the group consisting of a hematopoietic growth factor that promotes hematopoietic cell maintenance, expansion and/or differentiation, inoculated stromal cells and stromal cell conditioned medium.

10. A method for in vitro culture of hematopoietic progenitor cells to produce differentiated cells of hematopoietic origin comprising:
culturing, in a first culturing step, a first amount of hematopoietic progenitor cells in an environment that is free of inoculated stromal cells, stromal cell conditioned medium, and exogenously added hematopoietic growth factors that promote hematopoietic differentiation, other than serum, under conditions and for a period of time to increase the number or colony forming unit potential of hematopoietic progenitor cells relative to said first amount, thereby producing a second amount of hematopoietic progenitor cells, and then, in a second culturing step, culturing at least a portion of the second amount of hematopoietic progenitor cells in an environment that includes at least one of an agent selected from the group consisting of a hematopoietic growth factor that promotes hematopoietic cell maintenance, expansion and/or differentiation, inoculated stromal cells and stromal cell conditioned medium, to produce differentiated cells of hematopoietic origin.

11. The method of claim 10, wherein the environment of said first culturing step is free of interleukins 3, 6 and 11, Tpo, stem cell factor and FLT/FLK ligand growth factors.

12. The method of claim 10, wherein the environment is free of hematopoietic growth factors.

13. The method of claim 10, wherein the second culturing step is a plurality of second culturing steps, each comprising culturing only a portion of said second amount of hematopoietic progenitor cells.

14. The method of claim 10, further comprising a harvesting step between said first and second culturing steps, wherein the harvesting step comprises harvesting the at least a portion of the second amount prior to culturing the at least a portion of the second amount in the second culturing step.

15. The method of claim 14, wherein said harvesting step comprises a plurality of harvesting steps spaced apart in time and wherein said second culturing step comprises a plurality of second culturing steps, one for each of said harvesting steps.

16. The method of claim 10, wherein said hematopoietic progenitor cells are obtained from a blood product.

17. The method of claim 16, wherein said blood product is mobilized peripheral blood or mobilized umbilical cord blood.

18. A method for in vitro culture of hematopoietic progenitor cells to produce differentiated cells of hematopoietic origin comprising:
culturing, in a first culturing step, hematopoietic progenitor cells in an environment that is free of inoculated stromal cells, stromal cell conditioned medium, and exogenously added hematopoietic growth factors that promote hematopoietic cell maintenance, expansion and/or differentiation, other than serum, to generate cultured hematopoietic progenitor cells,
intermittently harvesting only a portion of said cultured hematopoietic progenitor cells, to generate a plurality of intermittently harvested portions of cultured hematopoietic cells,
culturing, in a plurality of second culturing steps, the plurality of intermittently harvested portions, the second culturing steps carried out in an environment that includes at least one agent selected from the group consisting of a hematopoietic growth factor that promotes hematopoietic cell maintenance, expansion and/or differentiation, inoculated stromal cells and stromal cell conditioned medium, to produce differentiated cells of hematopoietic origin.

19. The method of claim 18, wherein the environment of said first culturing step is free of interleukins 3, 6 and 11, Tpo, stem cell factor and FLT/FLK ligand growth factors.

20. The method of claim 18, wherein the environment is free of hematopoietic growth factors.
